(19) United States
(12) Patent Application Publication
(10) Pub. No.: US 2011/0045479 A1
(43) Pub. Date: Feb. 24, 2011

(54) Method For High Resolution Melt Genotyping

(75) Inventor: Andreas R. Tobler, Fremont, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/859,238

(22) Filed: Aug. 18, 2010

Related U.S. Application Data
(63) Continuation of application No. 12/264,193, filed on Nov. 3, 2008, now abandoned.

(51) Int. Cl. C12Q 1/68

(52) U.S. Cl. 435/6

(57) ABSTRACT

Various methods are described that provide for high resolution melt (HRM) genotyping. The embodiments include providing a locus specific primer and two allele specific primers each having a 5′ end with a short tail, providing a nucleic acid having a single nucleotide polymorphism (SNP) base located within 1-20 base pairs of the 3′ end of nucleic acid, hybridizing the locus specific primer and the allele specific primers to the nucleic acid, amplifying the sample using pyrophosphorolysis activated polymerization (PAP) PCR enzyme, and determining the Tm of the amplicons using HRM. In other embodiments, reactions mixtures and kits for HRM genotyping are provided and disclosed. These kits comprise a locus specific primer, one or more allele specific primers each having a 5′ end with a short tail, a nucleic acid, and a pyrophosphorolysis activate polymerization (PAP) PCR enzyme.

### CROSS REFERENCE TO RELATED APPLICATION

This application is a continuation of U.S. application Ser. No. 12/264,193 filed Nov. 3, 2008, which is hereby incorporated by reference in its entirety.

### BACKGROUND

Polymerase chain reaction (PCR) is a primer extension reaction that provides a method for amplifying specific nucleic acids in vitro. Generally, in PCR, the reaction solution is maintained for a short period at each of three temperatures, 96° C., 60° C. and 72° C., to allow strand separation or denaturation, annealing, and chain extension, respectively. These three temperatures stages are repeated over various multiple cycles with an automated thermocycler that can heat and cool rapidly. PCR is a particularly useful tool for studying and analyzing DNA sequence variations.

Methods for sequence variation can be divided into a few simple categories: 1) genotyping for a know sequence or variance; and 2) scanning for an unknown sequence or variance. Most scanning techniques for sequence variants require gel electrophoresis or column separation after PCR. In many cases these and other techniques slow down the analysis, provide for sample loss, or do not provide accurate results. Further, most of these techniques do not have the ability to resolve certain sequence variants.

More recently PCR has been combined with fluorescent dyes in order to more quickly and accurately resolve sequence variants. PCR combined with fluorescent dyes has been studied to provide for a simpler and efficient way to determine sequence variants in DNA. Various DNA amplicons combined with fluorescent dyes have been studied to determine sequence variants such as single nucleotide polymorphisms (SNP). Single nucleotide polymorphisms are by far the most common genetic variations observed in man and other species. In these polymorphisms, only a single base varies between individuals. The alteration may cause an amino acid change in a protein, alter rate of transcription, affect mRNA splicing, or have no apparent effect on cellular process. Various types of dyes have been useful for this process. Some dyes will bind to single stranded DNA, double stranded DNA or will intercalate into the base pairs of the DNA. Examples of dyes in present use include and are not limited to SYTO9®, Eva Green∩, Quantace, BEBO, SYBR® Green, and LC Green®.

Further, many of the fluorescent dye methods have been used successfully to distinguish SNP's. However, in many cases typically high resolution of the amplicon is not possible due to the inability to distinguish among small sequence variants.

High resolution melting (HRM) is a novel, homogeneous, close-tube, post-PCR method, enabling genomic researchers to analyze genetic variations (SNPs, mutations, methylations) in PCR amplicons. It goes beyond the typical classical melting curve analysis by allowing scientists the ability to study the thermal denaturation of a double-stranded DNA in much more detail and with much higher information yield than ever before. HRM characterizes nucleic acid samples based on their disassociation (melting) behavior. Samples can be discriminated according to their sequence, length, GC content or strand complementarity. Even single base changes such as SNPs (single nucleotide polymorphisms) can be readily identified.

The most important High Resolution Melting application is gene scanning—the search for the presence of unknown variations in PCR amplicons prior to or as an alternative to sequencing. Mutations in PCR products are detectable by High Resolution Melting because they change the shape of DNA melting curves. A combination of new-generation DNA dyes, high-end instrumentation and sophisticated analysis software allows to detect these changes and to derive information about the underlying sequence constellation.

High resolution melting (HRM) is a method that analyzes the melting of a PCR amplicon in the presence of a saturating intercalating DNA dye. The analysis of short fragments (60-100 base pairs) as well as longer (up to 400 base pairs) can be used to detect the genotype of a single nucleotide polymorphism (SNP). Generally differences in melting temperature (Tm) and curve shape are used to determine SNP genotypes.

The nature and type of SNPs has a large impact on the accuracy and sensitivity of the HRM assay. For instance, heterozygote genotypes are easier to identify because of the change in curve shape and/or Tm. In contrast, homozygote genotypes differ only in the Tm and not in their curve shape and are, therefore, more difficult to distinguish. In addition, not all homozygotes can be distinguished by Tm. In such cases, heteroduplex analysis is necessary for complete genotyping. The problem with most of the above described methods is that they are not universally applicable to a variety of situations or SNP types. In addition, many of the techniques lack the ability to distinguish homozygotes (base inversions). What is needed is a more universal method that can allow for HRM analysis of all SNP's with higher accuracy, independent of the nature of the SNP.

### SUMMARY

Various embodiments provide methods for high resolution melt (HRM) genotyping. The methods comprise providing a locus specific primer, providing two allele specific primers each having a 5′ end with a short tail, providing a nucleic acid having a SNP base located within 1-20 base pairs of the 3′ end of the nucleic acid, hybridizing the locus specific primer and the allele specific primers to the nucleic acid, amplifying the sample using PAP PCR, and determining the Tm of the amplicons using HRM. In other embodiments reaction mixtures and kits for HRM genotyping are provided. The reaction mixture for HRM genotyping comprises a locus specific primer and two allele specific primers each having a 5′ end having a short tail. The reaction mixtures may optionally comprise one or more nucleic acids or one or more PAP PCR enzymes.

The described kits comprise a locus specific primer, one or more allele specific primers each having a 5′ end with a short tail, a nucleic acid, and one or more PAP PCR enzymes.

These and other features of the present teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows a perspective view of a general thermocycler and HRM instrument used with the present embodiments.

FIG. 2 shows a flow chart of the general methods employed with the present embodiments.

FIG. 3 shows a diagram of the locus specific and allele specific primers and how they are used with PAP PCR to amplify nucleic acids for HRM analysis.

FIG. 4A shows various possible SNP case scenarios that may be detected using the HRM.

FIG. 4B shows the associated HRM melt curves for each of the SNP case scenarios in FIG. 4A.

### DESCRIPTION OF VARIOUS EMBODIMENTS

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. Thus for example, reference to a “primer” includes more than one “primer”, reference to “genomic DNA” may refer to more than one strand of “genomic DNA”. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

In describing and claiming the embodiments, the following terminology will be used with the definitions set out below.

The abbreviations for the various nucleic acid bases include guanine (G), thymine (T), adenine (A) and Cytosine (C).

The term “allele specific primer” refers to a primer that binds to a specific sequence (the minority of sequences belong to genes) on a region of a nucleic acid to be amplified. These types of primers are used to amplify and discriminate between two or more alleles of a gene simultaneously. The difference between the two alleles can be a SNP, insertion or deletion.

The term “amplicons” refers to portions of nucleic acid that are to be amplified or multiplied using the polymerase chain reaction methodology (PCR).

The term “computer” refers to all the associated hardware, processors and displays to perform data acquisition and analysis.

The term “genomic DNA” refers to the total DNA from an organism. The whole complement of an organism's DNA. Typically this includes both the intron and exon sequences and the non-coding regulatory sequences such as the promoter and enhancer sequences.

The term “high resolution melt (HRM)” refers to a technique using PCR and one or more nucleic acid binding dyes that allows for the determination of sequence variation in a nucleic acid.

The term “locus specific primer” refers to a primer that binds to a particular region of a nucleic acid to be amplified. Generally an allele specific and locus specific primer is required to perform PCR on leading and lagging strands of the DNA or the template strand and complement.

The term “nucleic acid” or “nucleic acid strand” refers to a DNA or cDNA or versions of the same produced or processed from any type of nucleic acid. For instance, DNA, cDNA, RNA, mRNA, tRNA or modified or derivitized versions of the same.

The term “primer” refers to an oligonucleotide or short single-stranded nucleic acid which, upon hybridization with a complementary portion of another single-stranded molecule, acts as a starting point for initiation of polymerization mediated by an enzyme with DNA polymerase activity. Most typing methods used in clinical or research laboratories are based on amplification of specific genes from genomic DNA using polymerase chain reaction (PCR). PCR amplification of genes involves the use of locus specific, group-specific, or allele-specific primers. Locus specific primers amplify all alleles encoded at a given locus but not alleles encoded by other loci. Allele specific primers amplify families of alleles that share a common polymorphism. Allele specific primers are used to amplify a single allele and can differentiate between two sequences that differ by only a single base change. Strategies for amplification can include combinations of locus specific primers to amplify and analyze both alleles in a heterozygous sample, followed by group-specific or allele specific amplification to isolate one of the two alleles for further characterization.

The term “PAP PCR enzyme” refers to any enzyme that can perform PAP polymerizations reactions (also called pyrophosphorolysis activate polymerization chain reaction).

The term “pyrophosphorolysis activate polymerization (PAP) (PAP refers to a reaction that works in a reverse reaction to DNA polymerization and results in the removal of the 3′ terminal nucleotide of an annealed oligonucleotide.

The term “single nucleotide polymorphism (SNP)” refers to a DNA sequence variation occurring when a single nucleotide—A, T, C, or G—in the genome (or other shared sequence) differs between members of a species (or between paired chromosomes in an individual). For example, two sequenced DNA fragments from different individuals, AAGCCTA to AAGCTTA, contain a difference in a single nucleotide. In this case we say that there are two alleles: C and T. Almost all common SNPs have only two alleles.

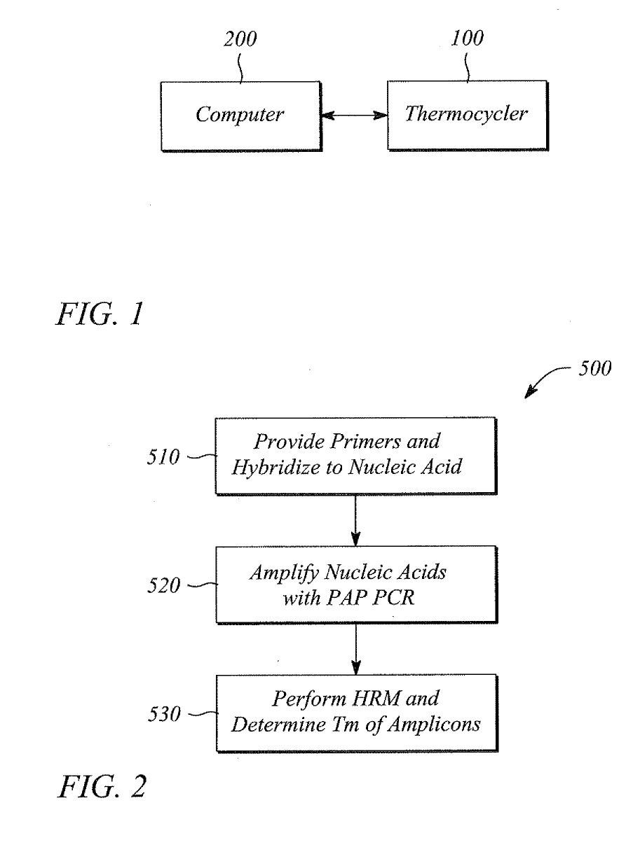

The embodiments are described with reference to the figures. In certain instances, the figures may not be to scale and have been exaggerated for clarity of presentation. In general it should be noted that allele specific PAP with tailed primers followed by HRM analysis turns HRM into an assay that can be applied to the analysis of any SNP, not just a subset of SNPs. Unexpectedly, it greatly increases the resolution of a SNP assay by adjusting alleles specifically to the length and sequence of a PCR amplicon. It further opens the opportunity to use the HRM assay in a quantitative way, e.g. for allele-quantification of SNPs, since the melt curves of the two amplicons are clearly separated. However, one disadvantage of HRM analysis is the intercalating DNA dye can not distinguish between specific and non-specific PCR amplification. Remarkably and unexpectedly, the high specificity of PAP-PCR greatly reduces the risk of non-specific PCR amplification and increased specificity as well as sensitivity of HRM assays. HRM-based sequence analysis is a powerful technology for SNP genotyping and mutation scanning. One problem HRM based assays face is that not all SNPs can be analyzed by HRM, and that assay reproducibility is low if the Tm difference between two PCR amplicons is small. Allele specific PAP PCR with tailed primers followed by HRM analysis addresses both of these issues. It converts the HRM platform into a robust and quantitative mutation screening platform capable of analyzing any SNP. An increased allele specific resolution between PCR amplicons also allows quantitative genotyping applications like allele quantitation, or allele specific gene expression analysis. A very robust assay platform is further necessary to design assays for clinical research as well as diagnostic applications. Having generally discussed the embodiments, a more detailed description is now in order. Referring now to FIG. 1, the embodiments will now be described in more detail.

FIG. 1 shows a real time thermocycler instrument 100 with high resolution melt capability (HRM) that may be used with the present embodiments. Various types of thermocyclers have been described in the literature to perform PCR. Some types of thermocyclers with HRM that may be employed with the present embodiments include and are not limited to the AB 7300, the HR-1™, the LightCycler 480®, the Master Cycler®, the LightScanner® and the Rotor-Gene™. Each of these instruments typically provides a real time PCR reaction followed by HRM. The thermocycler 100 may be employed with various types of computers 200 or software 300. The computers 200 and software 300 may be employed for various HRM analyses. Generally the thermocycler 100 performs a number of PCR amplification reactions. After these reactions have been completed the results are subjected to HRM to generate a melt curve. The HRM melt curve is typically displayed on the computer 200 or other similar type device with user interface. The data and results are calibrated and displayed using software 300. The software 300 may be present in computer 200 or on a computer readable medium.

When it comes to genotyping and mutation scanning, HRM is emerging as the technique of choice because it is inexpensive simple, accurate and rapid. Development of this method of DNA analysis has been underway since its introduction in 2002. The first high-resolution instrument developed, provide for accuracy and high throughput. In addition to the special instrumentation, high-resolution melting uses special saturation dyes that fluoresce only in the presence of double stranded DNA. These dyes are included in the PCR amplification process. When the sample is heated to high temperatures, the DNA denatures and the fluorescent color fades away as the double stranded DNA separates, generating a melting curve. Because different genetic sequences melt at slightly different rates, they can be viewed, compared, and detected using these curves. Even a single base change will cause differences in the melting curve. The process can be used for specific genotyping, comparing sequence identity between two DNA samples, and scanning for any sequence variant between two primers. High-resolution DNA melting is becoming more popular as its accuracy and simplicity is recognized. High-resolution DNA melting makes it possible to quickly and accurately determine whether DNA sequences match, providing an interesting option for transplantation matching and forensics. Genotyping via high-resolution melting is more streamlined and less expensive than methods that use complex probes.

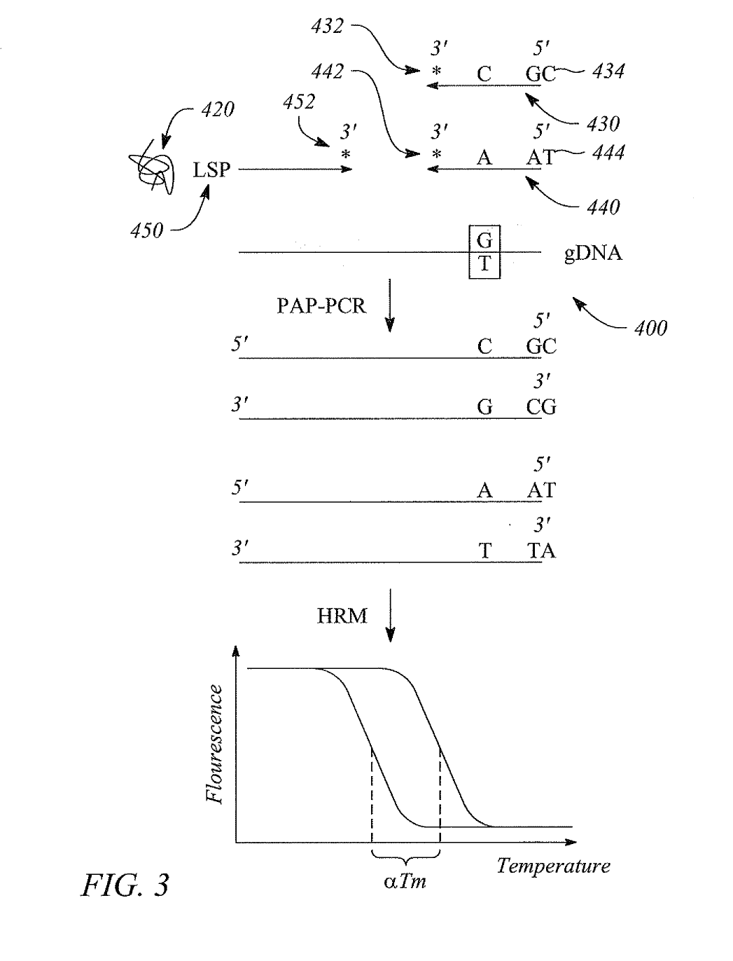

Referring now to FIGS. 2-3 the embodiments will now be discussed in more detail. FIG. 3 shows a nucleic acid 400 that may be used for PAP PCR. The nucleic acid 400 may comprise cDNA or DNA or versions of the same derived or processed from any type of nucleic acid. In certain instances it may comprise genomic DNA (gDNA as shown in the figure) from a single organism. In other cases it may comprise a mixture of nucleic acids or nucleic acids from various organisms. It should also be noted in the present embodiments that genotyping can be accomplished for both known and unknown portions of the nucleic acid. However, in most instances the SNP of interest is typically located in or around one of the specific primers being employed (See FIG. 3). In addition, typically the position of the SNP may or may not be known. In the present example a known G to T SNP is shown. For instance, the nucleic acid 400 shows a G to T SNP that is present near the 3′ end of the DNA (SNP is shown and marked in the block and donates a change from G to T). As provided in the embodiments various types of SNPs may be provided or present in the nucleic acid. Also, it is within the scope of the embodiments that more than one SNP may also be present. The SNP is typically located with 1-20 base pairs of the 3′ end of the nucleic acid to ensure allele specific amplification. This is not a requirement of the embodiments, but may be a limitation of the enzymes being employed. For instance, the PAP PCR enzyme has been shown to be allele specific and does not typically allow for PCR extension when there is a mismatch in base pairing. Further, the mismatch may occur within the strand length of the allele specific primer. For instance, in certain embodiments this would comprise the first 1-20 base pairs of the allele specific primer or the complement nucleic acid strand. The present embodiments exploit this enzyme specificity. It should also be noted that although a PAP PCR enzyme 420 may be employed with the present embodiments, other enzymes may also be possible. The important aspect of the enzyme being its capability of extending blocked primer ends only upon proper base pair matching in the nucleic acids and the SNP. Other embodiments that are similar or different to the PAP PCR enzyme are within the scope of the present embodiments.

FIG. 3 also shows a first allele specific primer 430 and a second allele specific primer 440 that are employed with the present embodiments. The first allele specific primer 430 and the second allele specific primer 440 may comprise any number of nucleotides and lengths. It is within the scope of the embodiments that various primer types may be employed with the present embodiments.

The first allele specific primer 430 comprises a blocked 3′ end 432. The blocked end 432 may be blocked in any number of different ways know in the art. This may be accomplished using chemical modification, based pair alteration etc. The blocked end 432 is designed to prevent normal PCR extension of the primer during amplification. For instance, the blocked end 432 may be a dideoxy end that is blocked from providing normal PCR extension and amplification.

The first allele specific primer 430 also comprises a 5′ end that comprises a first tail 434. The first tail 434 may comprise any desired number and types of nucleotide bases, or additions of any kind that change the Tm of the amplicon. In FIG. 3 the first tail 434 is show as a GC sequence. The sequence in certain embodiments may be many more nucleotides long. As will be discussed below the first tail 434 will be important in helping to distinguish the type of SNP present in the nucleic acid. This is mainly accomplished by the different Tm's that have been determined using HRM.

The first allele specific primer 430 may comprise a known nucleotide position shown as C that is used to probe for a particular SNP alteration in the nucleic acid. For instance, in this case the know alteration would be to G in the nucleic acid or gDNA (as shown in FIG. 3).

The second allele specific primer 440 comprises a blocked 3′ end 442. The blocked end 442 (all blocked ends are shown in the FIGS. with a *) may be blocked in any number of different ways known in the art. This may be accomplished using chemical modification, base pair alteration etc. The blocked end 442 is designed to prevent normal PCR extension of the primer during amplification. For instance, the blocked end 442 may be a dideoxy end that is blocked from providing normal PCR extension and amplification.

The second allele specific primer 440 also comprises a 5′ end that comprises a second tail 444. The second tail 444 may comprise any desired number and type of nucleotide bases, or additions of any kind that change the Tm of the amplicon. It should be noted that in certain embodiments the second tail 444 of second allele specific primer 440 may differ in length or nucleotide sequence from the first tail 434 of the first allele specific primer 430. In FIG. 3 the second tail 444 is show as an AT sequence. The sequence in certain embodiments may be many more nucleotides long. As will be discussed below the second tail 444 may be important in helping to distinguish the type of SNP present in the nucleic acid. This is mainly accomplished by the difference in Tm.

The second allele specific primer 440 may comprise a known nucleotide position shown as C (in FIG. 3) that may be used to probe for a particular SNP alteration in the nucleic acid. For instance, in this case the known alteration would be to G in the nucleic acid (shown as a gDNA).

FIG. 3 also shows the presence of a locus specific primer 450 having a blocked end 452. The locus specific primer 450 binds to a particular location or locus in close proximity to the first allele specific primer 430 or the second allele specific primer 440. In either case typically a second primer such as the locus specific primer 450 is necessary in order to allow for PCR extension of one or more of the nucleic acid strands. The locus specific primer 450 may comprise any number of sequences or sequence lengths. It is within the scope of the embodiments to provide various types of locus specific primers of varying length or sequence. The blocked end 452 (all blocked ends are shown in the FIGS. with a *) may be blocked in any number of different ways known in the art. This may be accomplished using chemical modification, base pair alteration etc. The blocked end 452 is designed to prevent normal PCR extension of the primer during amplification. For instance, the blocked end 452 may be a dideoxy end that is blocked from providing normal PCR extension and amplification.

The first allele specific primer 430, the second allele specific primer 440, the locus specific primer 450 and an optional nucleic acid 400 (DNA, cDNA, or versions of the same derived or processed from any type of nucleic acid) may be combined together to make a kit 460 (kit not shown in FIGS). The kit can be designed in any number of ways or combinations.

Also, it is within the scope of the embodiments that certain reaction mixtures may also be provided that comprise various combinations of the locus specific primer 450, the first allele specific primer 430 and the second allele specific primer 440. An optional nucleic acid 400 may also be present in the reaction mixtures.

It should be noted that about 84% of all human SNP's result in A:T to G:C interchange with a Tm difference of approximately 1° C. in small amplicons. In 16% of SNP's the base pair is inverted (A:T to T:A, or G:C to C:G) and the Tm difference is smaller with a Tm at about 0.1° C. However, a robust HRM assay should have a large Tm difference between genotypes, and it should be capable of analyzing all SNP's. This can be achieved by performing an allele specific PAP PCR with tailed primers followed by HRM analysis (as described herein).

It should be noted that Pyrophosphorolysis PCR (PAP PCR) is the reverse reaction of DNA polymerization and results in the removal of the 3′ terminal nucleotide of an annealed oligonucleotide. Primers used for PAP-PCR are blocked at their 3′ end and have to be activated by pyrophosporolysis for extension to occur. The activation of a 3′ blocked primer is a very specific event, since mismatches occur not only at the 3′ end, but within the primer. For example in at least 1 to 20 base pairs of the 3′ end essentially block activation. This property can be exploited to increase the Tm difference between two amplicons in an allele specific way.

Having discussed the general embodiments, the components of the embodiments and the reaction mixtures, a description of the general methods are now in order.

Referring now to FIG. 2, a flow chart of the methods is shown. The general method for HRM 500 comprises providing primers and hybridizing to a nucleic acid 510, amplifying nucleic acids with a PAP PCR enzyme 520 and performing HRM to determine Tm of amplicons 530. Further details of the method will now be described.

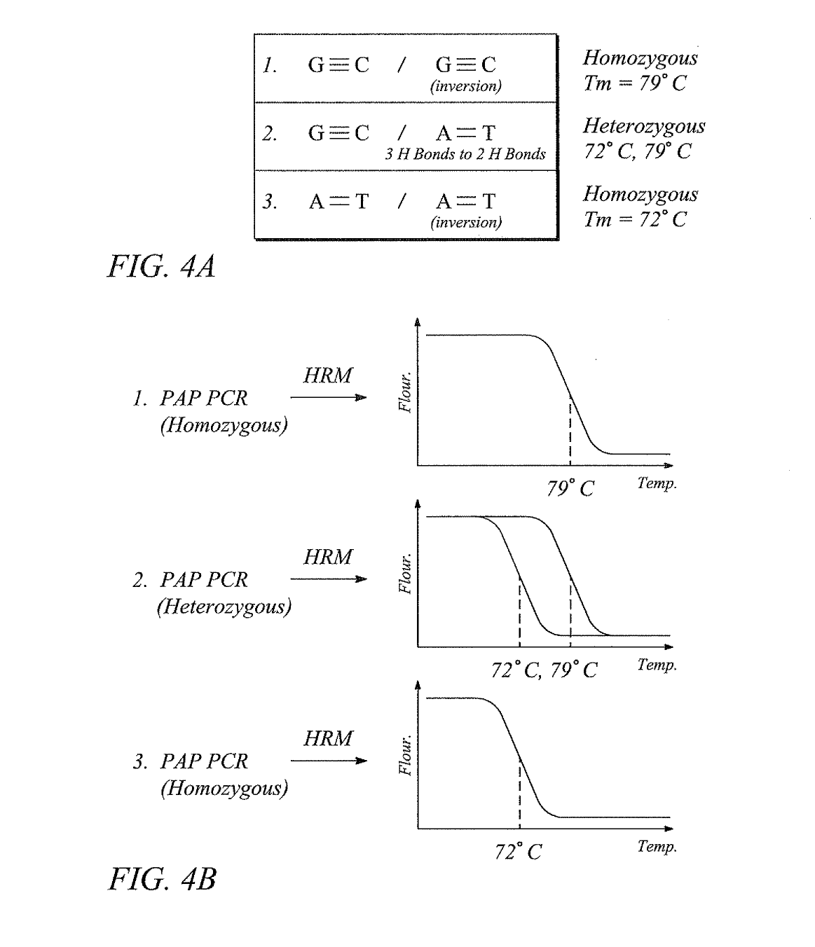

Referring now to FIG. 3-4, the nucleic acid 400 is shown with a G to T SNP located proximal to the 3′ end of the strand. SNP's may be either homozygous or heterozygous in origin. For instance, when a nucleotide changes from A:T to T:A or G:C to C:G we say that the SNP is homozygous (or the base is inverted). When the base pair change is G:C to A:T or similar type change of a purine (guanine and adenine are purines and thymine and cytosine are pyrimidines) base, the result is a change in hydrogen bonding (i.e. from three bonds to two in this case or vice versa). This change in hydrogen bonding impacts the overall Tm and results in two different melting curves as shown in FIG. 4. The curves show Tm curves at 72° C. and 79° C. (called a heterozygous base pairing). Note that the G:C to C:G base inversion is distinguishable from the A:T to T:A base inversion by the difference in the Tm. The Tm for the G:C inversion is shown in FIG. 4 as being at 79° C. The A:T to T:A inversion has a Tm at 72° C. Therefore, both Tm and curve shape are important for identification and analysis. Homozygous SNP inversions are typically more difficult to distinguish since the ATM differs in only about 0.1° C. Whereas, the heterozygous inversion is typically more easily distinguishable with a ATM of about 1.0° C. The present embodiments can be used to easily distinguish these situations. For instance, the use of the first tail 434 on the first allele specific primer 430 and the second tail 444 on the second allele specific primer 440 provides the ability to distinguish one situation from the next. In other words, the first tail 434 and the second tail 444 effect the overall Tm of the HRM in such a way that the overall ATM is increased (or more easily distinguishable). This makes it easier to distinguish which situation is present with each SNP. For simplicity FIG. 3 shows the heterozygous SNP from G to T in order to make it easier to show the overall PAP PCR methods and embodiments.

We claim:

1. A method for high resolution melt (HRM) genotyping, comprising:
(a) providing a locus specific primer;
(b) providing a first allele specific primer and a second allele specific primer, each allele specific primer having a 5′ end with a short tail;
(c) providing a nucleic acid having a SNP base located within 1-20 base pairs of the 3′ end of the nucleic acid;
(d) hybridizing the locus specific primer and the allele specific primers to the nucleic acid;
(e) amplifying the nucleic acid using pyrophosphorolysis activated polymerization (PAP) PCR; and
(f) determining the Tm of the amplicons using high resolution melt analysis.

2. A method as recited in claim 1, wherein the single nucleotide polymorphism (SNP) genotype alteration is a heterozygote.

3. A method as recited in claim 1, wherein the single nucleotide polymorphism (SNP) genotype is a homozygote.

4. A method as recited in claim 1, wherein the single nucleotide polymorphism (SNP) comprises a G to T change.

5. A method as recited in claim 1, wherein the single nucleotide polymorphism (SNP) comprises an A to C change.

6. A method as recited in claim 1, wherein the single nucleotide polymorphism (SNP) comprises a T to G change.

7. A method as recited in claim 1, wherein the single nucleotide polymorphism (SNP) comprises a C to A change.

8. A method as recited in claim 1, wherein the sample is amplified using a pyrophosphorolysis activated polymerization (PAP) PCR enzyme.

9. The method of claim 1, wherein both 5′ ends of the allele specific primers comprise short tails.

10. The method of claim 1, wherein at least one 5′ end of an allele specific primer comprises a short tail.

11. A method as recited in claim 1, wherein the allele specific primer short tail comprises GC.

12. A method as recited in claim 1, wherein the allele specific primer short tail comprises AT.

13. A method as recited in claim 1, wherein the amplification step is performed using a PCR thermocycler.

14. A method as recited in claim 1, wherein the difference in Tm and curve shape are used to determine the single polynucleotide polymorphism (SNP) genotype.

15. A method as recited in claim 1, wherein the nucleic acid strand comprises 1-60 bases pairs.

16. A method as recited in claim 1, wherein the nucleic acid strand comprises 1-1000 bases pairs.

17. A kit for high resolution melt (HRM) genotyping, comprising:
(a) a locus specific primer;
(b) one or more allele specific primers having a 5′ end with a short tail;
(c) a nucleic acid; and
(d) a pyrophosphorolysis activated polymerization (PAP) PCR enzyme.

18. A reaction mixture for HRM genotyping, comprising:
(a) a locus specific primer; and
(b) one or more allele specific primers having a short tail.

19. The reaction mixture as recited in claim 17, further comprising a nucleic acid.

20. The reaction mixture of claim 18, wherein the nucleic acid comprises DNA or cDNA.

21. A reaction mixture as recited in claim 18, further comprising a pyrophosphorolysis activated polymerization (PAP) PCR enzyme.
